(19) United States
(12) Patent Application Publication
(10) Pub. No.: US 2026/0257225 A1
(43) Pub. Date: Sep. 3, 2026

(54) COLORED COVERLAY DIELECTRICS IN DIGITAL MICROFLUIDICS

(71) Applicant: Nicoya Lifesciences Inc., Kitchener (CA)

(72) Inventors: Harish KRISHNAKUMAR, Kitchener (CA); Arjun SUDARSAN, Carlsbad, CA (US)

(21) Appl. No.: 18/839,345

(22) PCT Filed: Jun. 9, 2023
(86) PCT No.: PCT/CA2023/050801

§ 371 Date: Aug. 16, 2024

Related U.S. Application Data
(60) Provisional application No. 63/350,754, filed on Jun. 9, 2022.

(51) Int. Cl. B01L 3/00

(52) U.S. Cl. CPC B01L 3/502784; B01L 2200/028; B01L 2200/16; B01L 2300/0627; B01L 2300/0887; B01L 2300/12; B01L 2300/168

(57) ABSTRACT

Provided herein are devices, systems and methods comprising a digital microfluidics (DMF) cartridge, the DMF cartridge comprising: a top plate, wherein the top plate comprises a top plate substrate; a bottom plate, wherein the bottom plate comprises a colored coverlay substrate; and a droplet operation gap between the top plate and the bottom plate.

### CROSS-REFERENCE

This application claims the benefit of U.S. Provisional Application No. 63/350,754, filed Jun. 9, 2022, which is hereby incorporated by reference in its entirety herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BACKGROUND

The inventive concepts described herein are directed to devices, systems and methods for improved detection of an analyte of interest in a sample droplet. More specifically, the present inventive concepts are directed to devices, systems and methods for using colored coverlay dielectrics for the improved detection of analytes (e.g., chemical or biochemical) on a digital microfluidics platform.

In digital microfluidics (DMF), it is possible to measure reflectance, fluorescence, chemiluminescence and/or evanescent-waves, for example, by using ultraviolet (UV), visible, infrared radiation (IR) or terahertz electromagnetic radiation for interrogation of an analyte-ligand interaction in a sample. For example, reflected or scattered light may be measured to quantify interactions between a ligand and an analyte. In addition, reflected or scattered light may be measured to detect a reporter moiety in enzyme linked immunosorbent assays (ELISAs) (which may be quantified via absorbance or optical density). However, many assay types, such as assays with colorimetric readouts of transparent or semi-transparent samples, can result in a poor detection signal or a poor readout due to low visual contrast between the signal and the sample. The resulting low visual contrast and poor readout can complicate the detection of the target analyte in the assay, which may lead to a higher limit of detection (LOD) and/or reduced assay sensitivity.

Accordingly, there is a need in the art for improved devices, systems and methods for detecting analytes and/or bio-molecular interactions in aqueous samples. To meet this need, the present inventive concepts described herein provide for DMF cartridges, devices, methods and systems that use a colored coverlay of dielectrics for improved interrogation and analysis of aqueous samples (or aqueous droplets in DMF devices).

### SUMMARY

Provided herein are microfluidics devices comprising a digital microfluidics (DMF) cartridge, the DMF cartridge comprising: a top plate, wherein the top plate comprises a top plate substrate; a bottom plate, wherein the bottom plate comprises a bottom plate substrate and a colored coverlay substrate; and a droplet operation gap between the top plate and the bottom plate.

In some embodiments, the bottom plate substrate comprises a printed circuit board (PCB) substrate, a glass substrate, a silicon substrate, or a polyamide substrate, and wherein the PCB substrate, the glass substrate, the silicon substrate, or the polyimide substrate further comprises the colored coverlay substrate.

In some embodiments, the top plate substrate comprises a printed circuit board (PCB) substrate, a glass substrate, a silicon substrate, or a polyamide substrate, and wherein the PCB substrate, the glass substrate, the silicon substrate, or the polyimide substrate further comprises the colored coverlay substrate.

In some embodiments, the color of the colored coverlay substrate is white, light gray, silver, orange, light yellow, dark gray, near-black or black.

In some embodiments, the color of the colored coverlay substrate is white, light gray, silver, or light yellow.

In some embodiments, the white, light gray, silver, light yellow color of the colored coverlay substrate comprises a Hue, Saturation, and Value (HSV).

In some embodiments the HSV comprises a hue of from about 0 to about 70, a saturation of from about 0 to about 10%, and/or a value of from about 50% to about 100%.

In some embodiments the color of the colored coverlay substrate is black.

In some embodiments the dark gray, near-black or black color of the colored coverlay substrate comprises a HSV comprising a hue of from about 0 to about 80, a saturation of from about 0 to about 25%, and/or a value of from about 0% to about 50%.

In some embodiments the colored coverlay substrate comprises multiple colors on a surface of the colored coverlay substrate, and wherein each of the multiple colors are selected from white, gray, silver, light yellow, and black.

In some embodiments the multiple colors are sectioned on the surface of the colored coverlay substrate according to the assay being performed on the DMF cartridge.

In some embodiments the multiple colors are sectioned on the surface of the colored coverlay substrate to correspond to functions performed on the top plate and bottom plate.

In some embodiments each of the functions performed on the top plate and the bottom plate correspond to a color of the multiple colors.

In some embodiments the surface of the colored coverlay substrate comprises one or more regions.

In some embodiments the one or more regions each comprise one or more of the multiple colors.

In some embodiments the colored coverlay substrate comprises a multiple color coverlay substrate, and wherein the multiple colored coverlay substrate comprises a white section and a black section.

In some embodiments the droplet operation gap between the top plate and the bottom plate is filled with a filler fluid.

In some embodiments the filler fluid is a low-viscosity oil or a halogenated oil.

Provided herein are methods for analyzing an analyte of interest using an optical detection operation, the method comprising: (a) providing a digital microfluidic (DMF) cartridge, where the DMF cartridge comprises: (i) a top plate, wherein the top plate comprises a top plate substrate; (ii) a bottom plate, wherein the bottom plate comprises a colored coverlay substrate having a plurality of electrodes operable to perform droplet operations; and (iii) a droplet operation gap between the top plate and the bottom plate; (b) providing a sample droplet to be analyzed for the presence of the analyte of interest; (c) performing droplet operations on the droplet sample, wherein the performing comprises moving the droplet sample to a sensor well in the DMF cartridge; (d) performing an optical detection operation by illuminating the droplet sample in the sensor well using an illumination source; (e) detecting light emitting from the droplet sample using an optical measurement device; and (f) using a processor, detecting the presence of an analyte and/or determining the concentration of the analyte by analyzing the detected light.

In some embodiments, the optical detection operation comprises one or more of absorbance-based, colorimetric-based, luminescence-based, and/or fluorescence-based optical detection operations.

In some embodiments, the colorimetric-based optical detection comprises detecting light in the Cyan, Magenta, Yellow, and Key (CMYK) color space.

In some embodiments, the colorimetric-based optical detection comprises detecting light in the HSV color space.

In some embodiments, the method further comprises performing an enzyme-linked immunosorbent assay (ELISA) or a plasmonic enzyme-linked immunosorbent assay (pELISA).

In some embodiments, detecting the presence of the analyte and/or determining the concentration of the analyte comprises detecting a color change in a reporter moiety.

In some embodiments, the reporter moiety comprises 3,3′,5,5′-Tetramethylbenzidine (TMB), 2,2′-Azinobis [3-ethylbenzothiazoline-6-sulfonic acid]-diammonium salt (ABTS), o-phenylenediamine dihydrochloride (OPD), p-nitrophenyl phosphate (PNPP), or o-nitrophenyl-β-D-galactopyranoside (ONPG).

In some embodiments, the reporter moiety comprises gold, silver, platinum, palladium, copper, gold coated silver, gold coated nanoparticles, or silver coated nanoparticles.

In some embodiments, the reporter moiety comprises gold nanourchins.

In some embodiments, the color of the colored coverlay substrate is tuned to maximize the dynamic range and/or linear range of detection for one or more reporter moieties.

In some embodiments, the color of the colored coverlay substrate is tuned to maximize the limit of detection (LOD) for one or more reporter moieties.

In some embodiments, the color of the colored coverlay substrate is tuned to maximize the assay sensitivity.

In some embodiments, the color of the colored coverlay substrate is selected from white, light gray, silver, light yellow, dark gray, near-black and black.

In some embodiments, the color of the colored coverlay substrate is white, light gray, silver, or light yellow.

In some embodiments, the color of the colored coverlay substrate comprises a Hue, Saturation, and Value (HSV) comprising a hue of from about 0 to about 70, a saturation of from about 0 to about 10%, and/or a value of from about 50% to about 100%.

In some embodiments, the color of the colored coverlay substrate is dark gray, near-black, or black.

In some embodiments, the dark gray, near-black or black color of the colored coverlay substrate comprises a HSV comprising a hue of from about 0 to about 80, a saturation of from about 0 to about 25%, and/or a value of from about 0% to about 50%.

In some embodiments, the colored coverlay substrate comprises multiple colors, and wherein each of the multiple colors are selected from white, gray, silver, light yellow, dark gray, near-black and black.

In some embodiments, the colored coverlay substrate comprises a multiple color coverlay substrate, and wherein the multiple colored coverlay substrate comprises a white, light gray, silver or light yellow section and a dark gray, near-black or black section, and wherein steps (d) and (e) are performed in each of the white, light gray, silver or light yellow section and the dark gray, near-black or black section.

Provided herein are methods for detecting an analyte using colorimetric-based optical detection operations, the method comprising: (a) providing a digital microfluidic (DMF) cartridge, where the DMF cartridge comprises: (i) a top plate, wherein the top plate comprises a top plate substrate; (ii) a bottom plate, wherein the bottom plate comprises a colored coverlay substrate having a plurality of electrodes operable to perform droplet operations, wherein the color of the colored coverlay substrate is white, light gray, silver or light yellow; and (iii) a droplet operation gap between the top plate and the bottom plate; (b) providing a sample droplet to be analyzed for the presence of an analyte of interest; (c) performing droplet operations on the sample droplet to move the sample droplet to a sensor well in the DMF cartridge; (d) performing a colorimetric-based optical detection operation by illuminating the sample droplet using a colorimetric-based illumination source; (e) detecting light emitted from the sample droplet at one or more color-specific wavelengths using an optical measurement device; and (f) using a processor, detecting the presence of an analyte and/or determining the concentration of the analyte by analyzing the detected light.

In some embodiments, the colorimetric-based optical detection comprises detecting light in the CMYK color space.

In some embodiments, the colorimetric-based optical detection comprises detecting light in the HSV color space.

In some embodiments, the method further comprises performing an enzyme-linked immunosorbent assay (ELISA) or a plasmonic enzyme-linked immunosorbent assay (pELISA).

In some embodiments, detecting the presence of the analyte and/or determining the concentration of the analyte comprises detecting a color change in a reporter moiety.

In some embodiments, the reporter moiety comprises 3,3′,5,5′-Tetramethylbenzidine (TMB).

In some embodiments, the reporter moiety comprises gold, silver, platinum, palladium, copper, gold coated silver, gold coated nanoparticles, or silver coated nanoparticles.

In some embodiments, the reporter moiety comprises gold nanourchins.

In some embodiments, the color of the colored coverlay substrate is tuned to maximize the dynamic range and/or linear range of detection for one or more reporter moieties.

In some embodiments, the color of the colored coverlay substrate is tuned to maximize the limit of detection (LOD) for one or more reporter moieties.

In some embodiments, the color of the colored coverlay substrate is tuned to maximize the assay sensitivity.

In some embodiments, the color of the colored coverlay substrate is white, light gray, silver or light yellow.

In some embodiments, the white, light gray, silver or light-yellow color of the colored coverlay substrate comprises a Hue, Saturation, and Value (HSV) comprising a hue of from about 0 to about 70, a saturation of from about 0 to about 10%, and/or a value of from about 50% to about 100%.

In some embodiments, the colored coverlay substrate comprises multiple colors, and wherein each of the multiple colors are selected from white, light gray, silver, and light yellow.

In some embodiments, the colored coverlay substrate comprises a multiple color coverlay substrate and wherein the multiple colored coverlay substrate comprises a white, light gray, silver or light-yellow section and a dark gray, near-black or black section, and wherein steps (d) and (e) are performed in each of the white, light gray, silver or light-yellow section and the dark gray, near-black or black section.

Provided herein are methods for detecting an analyte using fluorescence-based optical detection operations, the method comprising: (a) providing a digital microfluidic (DMF) cartridge, where the cartridge comprises: (i) a top plate, wherein the top plate comprises a top plate substrate; (ii) a bottom plate, wherein the bottom plate comprises a colored coverlay substrate comprising a plurality of electrodes operable to perform droplet operations, wherein the color of the colored coverlay substrate is dark gray, near-black or black; and (iii) a droplet operation gap between the top plate and the bottom plate; (b) providing a sample droplet to be analyzed for the presence of an analyte of interest; (c) performing droplet operations on the sample droplet to move the sample droplet to a sensor well in the DMF cartridge; (d) performing fluorescence-based optical detection operations by illuminating the sample droplet using a fluorescence-based illumination source; (e) detecting a fluorescence signal emitted from the sample droplet using an optical measurement device; and (f) using a processor, detecting the presence of an analyte and/or determining the concentration of the analyte by analyzing the detected fluorescence signal.

In some embodiments, the fluorescent-based optical detection comprises detecting a fluorescent reporter moiety or an intensity change in a fluorescent reporter moiety.

In some embodiments, the color of the colored coverlay substrate is tuned to maximize the dynamic range and/or linear range of detection for one or more reporter moieties.

In some embodiments, the color of the colored coverlay substrate is tuned to maximize the limit of detection (LOD) for one or more reporter moieties.

In some embodiments, the color of the colored coverlay substrate is tuned to maximize the assay sensitivity.

In some embodiments, the dark gray, near-black or black color of the colored coverlay substrate comprises a Hue, Saturation, and Value (HSV) comprising a hue of from about 0 to about 80, a saturation of from about 0 to about 25%, and/or a value of from about 0% to about 50%.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 illustrates an exemplary digital microfluidics (DMF) system that can be used for optical analysis of a sample fluid in accordance with the present inventive concepts.

FIG. 2 illustrates an exemplary digital microfluidics (DMF) cartridge for optical analysis of a sample fluid containing a target analyte.

FIG. 3 illustrates a method for optical analysis of an analyte of interest in a droplet using an optical detection operation, in accordance with one embodiment of the present inventive concepts.

FIG. 4 illustrates a method for optical analysis of an analyte of interest in a droplet using a colorimetric-based optical detection operation, in accordance with one embodiment of the present inventive concepts.

FIG. 5 illustrates a method for optical analysis of an analyte of interest in a droplet using a fluorescence-based optical detection operation, in accordance with one embodiment of the present inventive concepts.

FIG. 6A is an image showing cyan counts (in CMYK color model) of various concentrations of oxidized TMB (TMB⁺) on white coverlay dielectric.

FIG. 6B is a graph showing cyan counts (in CMYK color model) of various concentrations of oxidized TMB (TMB⁺) on white coverlay dielectric.

FIG. 7A is an image of etched (using TMB⁺) gold nanourchins (AuNUs) on orange coverlay dielectrics.

FIG. 7B is an image of unetched gold nanourchins (AuNUs) on white coverlay dielectrics.

FIG. 8 is a graph showing the change in Hue of gold nanourchins on a white coverlay dielectric at various times in the presence of oxidized TMB (from 0 μM to 4 μM TMB⁺).

### DETAILED DESCRIPTION

In the following detailed description, reference is made to the accompanying figures, which form a part hereof. In the figures, similar symbols typically identify similar components, unless context dictates otherwise. The illustrative embodiments described in the detailed description, figures, and claims are not meant to be limiting. Other embodiments may be utilized, and other changes may be made, without departing from the scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

Although certain embodiments and examples are disclosed below, inventive subject matter extends beyond the specifically disclosed embodiments to other alternative embodiments and/or uses, and to modifications and equivalents thereof. Thus, the scope of the claims appended hereto is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments, however, the order of description should not be construed to imply that these operations are order dependent. Additionally, the structures, systems, and/or devices described herein may be embodied as integrated components or as separate components.

For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

The presently disclosed subject matter now will be described more fully hereinafter with reference to the accompanying drawings, in which some, but not all embodiments of the presently disclosed subject matter are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

### Digital Microfluidics (DMF) System

In some embodiments, a DMF system that may be used to practice the inventive concepts described herein may include a DMF cartridge, an optical detection system, and a controller. In some embodiments, the optical detection system may include, for example, an illumination source and an optical measurement device in relation to sensor elements. In some embodiments, the optical detection system may operate in absorbance mode. In other embodiments, the optical detection system may operate in transmission mode. In some embodiments, the controller may be configured to control fluid manipulation (e.g., droplet manipulation) by activating and/or deactivating electrodes (or pads) in the DMF cartridge. In some embodiments, the controller may be configured to manage the operations of the DMF system.

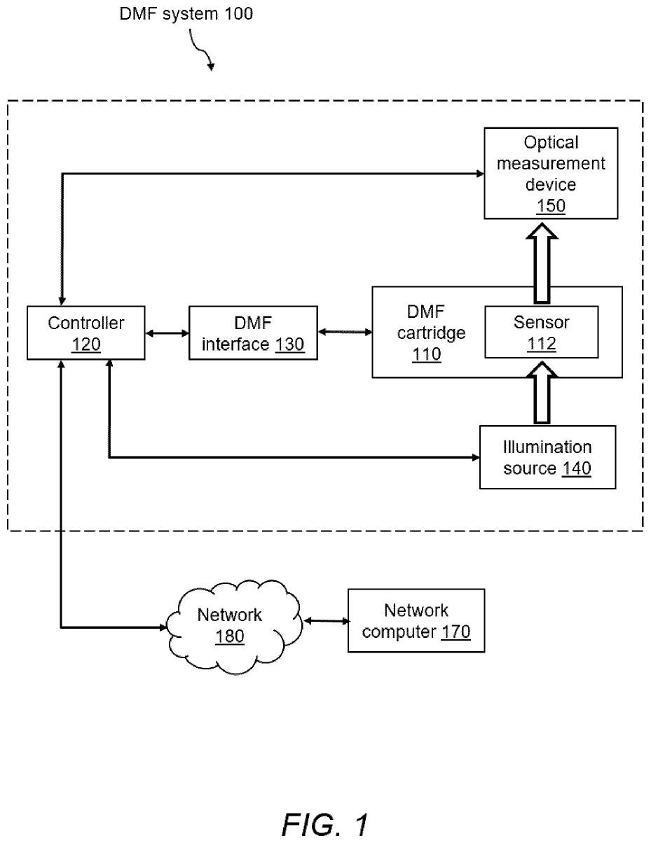

As shown in FIG. 1, in some embodiments, a DMF system 100 may be used. For example, FIG. 1 shows a block diagram of an embodiment of the presently disclosed DMF system 100 that may include a DMF cartridge 110. In some embodiments, the DMF cartridge 110 may include a sensor 112 for analysis of a target analyte. In some embodiments, the DMF system 100 may be used for analysis of a target analyte. In some embodiments, the analysis of the target analyte may include the detection, identification, quantification, or measuring of analytes. In some embodiments, the analysis of the target analyte may include the analysis of the interactions of the analytes with other substances (e.g., binding kinetics). In some embodiments, the analytes that may be analyzed may include, but are not limited to, small molecules, proteins, peptides, antibodies, nucleic acids, atoms, ions, polymers, or any combination thereof.

In some embodiments, DMF cartridge 110 may be configured to facilitate DMF capabilities such as fluidic actuation. For example, DMF cartridge 110 may facilitate droplet merging, droplet splitting, droplet dispensing, droplet diluting, or any combination thereof. In some embodiments, these DMF capabilities (e.g., droplet merging, droplet splitting, droplet dispensing, droplet diluting) can be used for sample preparation. For example, one non-limiting application of these DMF capabilities is fluid splitting of a ligand containing fluid. In this example, the DMF capabilities may allow a first portion of the ligand containing fluid to be used for ligand immobilization to the sensor and may allow a second portion of the ligand containing fluid to be used in a dissociation phase or dissociation step for determining a dissociation rate constant. In other embodiments, the DMF capabilities may be used for other processes, such as for example waste removal. For example, DMF cartridge 110 of DMF system 100 can be configured to be a disposable cartridge and/or a reusable cartridge.

In some embodiments, the use of a DMF system 100 comprising a DMF sensor 112 is used in the inventive concepts described herein. However, other systems and means for interrogating and analyzing a fluid can also be used in place of or in addition the DMF system disclosed herein. For example, other existing systems for absorbance (e.g., for measurements related to absorbed light) and/or transmission (e.g., for measurements of transmitted light) for interrogation of a fluid may be used. In some embodiments, the system used with the inventive concepts described herein is an electromagnetic radiation spectroscopy system, such as for example, ultraviolet-visible (UV-Vis) spectroscopy, Fourier-Transform Infrared (FTIR) spectroscopy, Raman spectroscopy, Circular Dichroism (CD) spectroscopy, Near-InfraRed (NIR) spectroscopy, Microfluidic Modulation spectroscopy (MMS), terahertz spectroscopy, or any combination thereof. In one embodiment, the system used with the inventive concepts described herein is an off-the-shelf electromagnetic radiation spectroscopy system. In some embodiments, the devices, DMF cartridges and methods described herein can leverage off-the-shelf plate readers (e.g., UV-Vis and FTIR spectrophotometers) for analysis of an analyte in a fluid.

In some embodiments, and as shown in FIG. 1, the DMF system 100 may further include a controller 120, a DMF interface 130, an illumination source 140, and an optical measurement device 150. In some embodiments, the controller 120 may be electrically coupled to the various hardware components of the DMF system 100, such as to the DMF cartridge 110, the illumination source 140, and the optical measurement device 150. In some embodiments, and as shown in FIG. 1, the controller 120 may be electrically coupled to the DMF cartridge 110 via the DMF interface 130, wherein the DMF interface 130 may be, for example, a pluggable interface that is configured to mechanically and electrically connect the DMF cartridge 110 and the controller 120.

In some embodiments, the controller 120 may be, for example, a general-purpose computer, a special purpose computer, a personal computer, a microprocessor, or another programmable data processing apparatus. In some embodiments, the controller 120 may be configured to provide processing capabilities, such as storing, interpreting, and/or executing software instructions, as well as controlling the overall operations of DMF system 100. In some embodiments, the software instructions may comprise a machine-readable code stored in a non-transitory memory that may be accessible by the controller 120 for the execution of the software instructions. In some embodiments, the controller 120 may be configured and programmed to control data and/or power aspects of the devices described herein. For example, with respect to DMF cartridge 110, the controller 120 may be configured to control fluid operations and/or droplet manipulation by activating and/or deactivating electrodes. In some embodiments, the controller 120 can be configured to provide functions of the DMF system 100. For example, the controller 120 can be used to authenticate the DMF cartridge 110 in a fashion that is similar to how printer manufacturers may check for their branded ink cartridges. In some embodiments, the controller 120 can be used to verify that the DMF cartridge 110 is not expired. In some embodiments, the controller 120 can be used to evaluate and/or confirm the cleanliness of the DMF cartridge 110 by running a certain protocol for that purpose.

Additionally, in some embodiments, the DMF cartridge 110 may include a capacitive feedback sensing (not shown). For example, a signal may be generated or detected by a capacitive sensor in the DMF cartridge 110 that can detect droplet position, velocity, and/or size. Further, in other embodiments, the DMF cartridge 110 may include a camera or other optical device that may provide an optical measurement of the droplet position, velocity, and/or size, and which can trigger the controller 120 to re-route the droplets at appropriate positions. In some embodiments, the feedback can be used to create a closed-loop control system to optimize droplet actuation rate and/or verify if droplet operations are completed successfully.

In some embodiments, and as shown in FIG. 1, the DMF system 100 may be connected to a network 180. For example, the controller 120 may be in communication with a networked computer 170 via the network 180. In some embodiments, the networked computer 170 can be, for example, any centralized server or cloud server. In some embodiments, the network 180 can be, for example, a local area network (LAN) or wide area network (WAN) for connecting to the internet.

In some embodiments, in the DMF system 100, an illumination source 140 and an optical measurement device 150 may be arranged with respect to a sensor 112 (e.g., fixed PR sensing and/or in-solution PR sensing) of a DMF cartridge 110. In some embodiments, the illumination source 140 may provide a light source comprising lights in the visible range (400-800 nm), such as, but not limited to, a white light-emitting diode (LED), a halogen bulb, an arc lamp, an incandescent lamp, lasers, or any combination thereof. In some embodiments, the illumination source 140 is not a white light source. In some embodiments, the illumination source 140 may provide any color of light that may be useful in the DMF system 100. In some embodiments, the optical measurement device 150 may be configured to obtain DMF light intensity readings for determining absorbance and/or transmission of light. In some embodiments, the optical measurement device 150 may be, for example, a charge coupled device, a photodetector, a spectrometer, a photodiode array, or any combinations thereof. Further, in some embodiments, the DMF system 100 is not limited to one illumination source 140 and one optical measurement device 150. For example, in some embodiments, the DMF system 100 may comprise one or more, two or more, three or more, four or more, five or more, six or more, seven or more, eight or more, nine or more, or 10 or more illumination sources 140. In some embodiments, the DMF system may comprise 10 or less, nine or less, eight or less, seven or less, six or less, five or less, four or less, three or less, or two or less illumination sources 140. In some embodiments, the DMF system 100 may comprise one or more, two or more, three or more, four or more, five or more, six or more, seven or more, eight or more, nine or more, or 10 or more optical measurement devices 150. In some embodiments, the DMF system may comprise 10 or less, nine or less, eight or less, seven or less, six or less, five or less, four or less, three or less, or two or less optical measurement devices 150.

In some embodiments, the illumination source 140 can be used for colorimetric-based optical detection operations. For example, the illumination source 140 may transmit light and the optical measurement device 150 may detect light in the CMYK color space or the Hue, Saturation, and Value (HSV) color space. In some embodiments, the DMF system 100 may include multiple illumination sources 140 and/or multiple optical measurement devices 150 to support multiple sensors.

### Devices, Methods and Systems for Analysis of a Sample Fluid

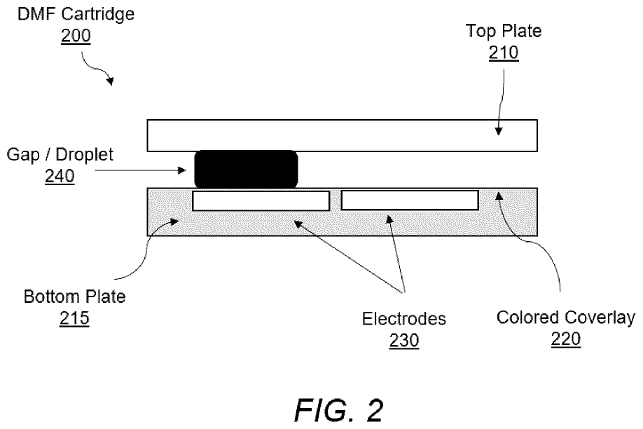

FIG. 2 illustrates an example of a detection device (e.g., a DMF cartridge) that may be used for optical analysis of a fluid (e.g., droplets) containing a target analyte. As shown in FIG. 2, the DMF cartridge 200 may include a top plate 210, a bottom plate 215 comprising a colored coverlay substrate 220, and a droplet operations gap 240 between the top plate 210 and the bottom plate 215. In some embodiments, the DMF cartridge 200 of the inventive concepts described herein may be configured to allow for multimodal measurements (e.g., obtaining measurements from either the top or sides of the DMF cartridge).

As shown in FIG. 2, the DMF cartridge 200 may comprise a top plate 210. In some embodiments, the top plate 210 may comprise a top plate substrate. In some embodiments, the DMF cartridge 200 may also comprise a bottom plate 215 that may comprise a colored coverlay substrate 220. In some embodiments, the bottom plate 215 comprising the colored coverlay substrate 220 may comprise a plurality of electrodes 230 that may be operable to perform droplet operations (e.g., droplet manipulation). FIG. 2 illustrates two electrodes 230 on bottom plate 215. However, the DMF cartridge may comprise more or less than two electrodes 230 on bottom plate 215. For example, in some embodiments, the DMF cartridge 200 may comprise one or more, two or more, three or more, four or more, five or more, six or more, seven or more, eight or more, nine or more, 10 or more, 11 or more, 12 or more, 13 or more, 14 or more, 15 or more, 16 or more, 17 or more, 18 or more, 19 or more, or 20 or more electrodes 230. In some embodiments, the DMF cartridge 200 may comprise 20 or less, 19 or less, 18 or less, 17 or less, 16 or less, 15 or less, 14 or less, 13 or less, 12 or less, 11 or less, 10 or less, nine or less, eight or less, seven or less, six or less, five or less, four or less, three or less, or two or less electrodes 230.

In some embodiments, the bottom plate 215 of the DMF cartridge 200 may comprises a colored coverlay substrate 220. In some embodiments, the colored coverlay substrate 220 may operates to enhance the visual contrast between a target analyte that may be present in the electrodes 230 and the DMF cartridge 200 itself. As such, in some embodiments, the colored coverlay substrate 220 may provide an improved or enhanced readout of the target analyte, which may improve the detection of the target analyte and the analysis of the target analyte.

In some embodiments, the colored coverlay substrate 220 may be used in applications using florescence-based measurements. For example, a black coverlay substrate may be used in florescence-based measurement applications to help in minimizing scattered and reflected light.

In some embodiments, the colored coverlay substrate 220 may be used in applications using luminescence-based measurements. For example, a white coverlay substrate may be used in luminescence-based measurement applications to increase reflections, which may improve the visualization of transparent droplets and luminescence-based measurements as compared to existing DMF cartridges that do not utilize a white colored coverlay substrate.

In some embodiments, the colored coverlay substrate 220 may include any color that may provide a contrast between a target analyte and the DMF cartridge 200 for detection of the targeted analyte. For example, in some embodiments, the color of the colored coverlay substrate 220 may include any color in the electromagnetic spectrum. In some embodiments, the color of the colored coverlay substrate 220 is black. In some embodiments, the color of the colored coverlay substrate 220 is white. In some embodiments, the color of the colored coverlay substrate 220 comprises a color on the visible light spectrum. In some embodiments, the color of the colored coverlay substrate 220 may comprise violet, indigo, blue, green, yellow, orange, red, or any combination thereof. In some embodiments, the color of the colored coverlay substrate 220 comprises a wavelength between about 380 nanometers (nm) to about 800 nm. In some embodiments, the color of the colored coverlay substrate 220 comprises a wavelength of 380 nm or more, 400 nm or more, 420 nm or more, 440 nm or more, 460 nm or more, 480 nm or more, 500 nm or more, 520 nm or more, 540 nm or more, 560 nm or more, 580 nm or more, 600 nm or more, 620 nm or more, 640 nm or more, 660 nm or more, 700 nm or more, 720 nm or more, 740 nm or more, 760 nm or more, 780 nm or more, or 800 nm or more. In some embodiments, the color of the colored coverlay substrate 220 comprises a wavelength of 800 nm or less, 780 nm or less, 760 nm or less, 740 nm or less, 720 nm or less, 700 nm or less, 680 nm or less, 660 nm or less, 640 nm or less, 620 nm or less, 600 nm or less, 580 nm or less, 560 nm or less, 540 nm or less, 520 nm or less, 500 nm or less, 480 nm or less, 460 nm or less, 440 nm or less, 420 nm or less, 400 nm or less, or 380 nm or less.

In some embodiments, the color of the colored coverlay substrate 220 may comprise a hue, saturation, and value (HSV).

In some embodiments, the color of the colored coverlay substrate 220 may comprise a hue. In some embodiments, the hue may be from about 0 to about 360. In some embodiments, the hue may be 0 or more, 20 or more, 40 or more, 60 or more, 80 or more, 100 or more, 120 or more, 140 or more, 160 or more, 180 or more, 200 or more, 220 or more, 240 or more, 260 or more, 280 or more, 300 or more, 320 or more, 340 or more, or 360 or more. In some embodiments, the hue may be 360 or less, 340 or less, 320 or less, 300 or less, 280 or less, 260 or less, 240 or less, 220 or less, 200 or less, 180 or less, 160 or less, 140 or less, 120 or less, 100 or less, 80 or less, 60 or less, 40 or less or 20 or less.

In some embodiments, the color of the colored coverlay substrate 220 may comprise a saturation. In some embodiments, the saturation may be from about 0% to about 100%. In some embodiments, the saturation may be 0% or more, 5% or more, 10% or more, 15% or more, 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, or 95% or more. In some embodiments, the saturation may be 100% or less, 95% or less, 90% or less, 85% or less, 80% or less, 75% or less, 70% or less, 65% or less, 60% or less, 55% or less, 50% or less, 45% or less, 40% or less, 35% or less, 30% or less, 25% or less, 20% or less, 15% or less, 10% or less, or 5% or less.

In some embodiments, the color of the colored coverlay substrate 220 may comprise a value. In some embodiments, the value may be from about 0% to about 100%. In some embodiments, the value may be 0% or more, 5% or more, 10% or more, 15% or more, 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, or 95% or more. In some embodiments, the value may be 100% or less, 95% or less, 90% or less, 85% or less, 80% or less, 75% or less, 70% or less, 65% or less, 60% or less, 55% or less, 50% or less, 45% or less, 40% or less, 35% or less, 30% or less, 25% or less, 20% or less, 15% or less, 10% or less, or 5% or less.

In some embodiments, the color of the colored coverlay substrate 220 can be selected from light colors, such as white, light gray, silver, or light yellow. In some embodiments, the color of the colored coverlay substrate 220 can be selected from dark colors, such as, dark gray, near-black or black. In some embodiments, the colored coverlay substrate 220 is white. In some embodiments, the colored coverlay substrate 220 is black.

In some embodiments, the colored coverlay substrate 220 may comprise a surface comprising multiple colors. For example, in some embodiments, the surface of the colored coverlay substrate 220 may comprise one or more, two or more, three or more, four or more, five or more, six or more, seven or more, eight or more, nine or more, 10 or more, 12 or more, 15 or more, 20 or more, 25 or more, 30 or more, 35 or more, 40 or more, 45 or more, 50 or more, 60 or more, 70 or more, 80 or more, 90 or more, or 100 or more colors. In some embodiments, the surface of the colored coverlay substrate 220 may comprise 100 or less, 90 or less, 80 or less, 70 or less, 60 or less, 50 or less, 45 or less, 40 or less, 35 or less, 30 or less, 25 or less, 20 or less, 15 or less, 12 or less, 10 or less, nine or less, eight or less, seven or less, six or less, five or less, four or less, three or less, two or less, or one or less colors.

In some embodiments, the multiple colors on the colored coverlay substrate may be sectioned on the surface of the colored coverlay substrate 220 according to the assay being performed on the DMF cartridge. For example, the multiple colors of the colored coverlay substrate may be sectioned according to absorbance-based optical detection operations, colorimetric-based optical detection operations, luminescence-based optical detection operations, fluorescence-based optical detection operations, or any combination thereof. In some embodiments, dark colors on the colored coverlay substrate may be used in some assays while light colors on the colored coverlay substrate may be used in other assays. In some embodiments, the multiple colors of the colored coverlay substrate may sectioned on the surface of the colored coverlay substrate to correspond to functions performed on the top plate and the bottom plate. In some embodiments, each of the functions performed on the top plate and the bottom plate may correspond to a color. In some embodiments, the surface of the colored coverlay substrate may comprise one or more regions. In some embodiments, the one or more regions may comprise one or more of the multiple colors. For example, a colored coverlay substrate may comprise five regions where each region of the five regions is a different color. As another example, a colored coverlay substrate may comprise 10 regions where 5 of the ten regions are one color, and the other 5 of the 10 regions are a different color.

In some embodiments, the colored coverlay substrate 220 may be used in assays using colorimetric-based measurements. For example, assays using colorimetric-based measurements may use a colorimeter (e.g., a spectrophotometer) to determine a concentration of a chemical compound in a solution by measuring a spectral absorbance of the chemical compound at a certain wavelength where a color may be formed during the reaction of a detection chemical with the target compound. For assays using colorimetric-based measurements, the color of the colored coverlay substrate 220 may comprise light colors, such as white, light gray, silver, light yellow, or any other light color. For example, the color of the colored coverlay substrate 220 may comprise a hue, saturation, and value (HSV) amount comprising a hue of from about 0 to about 70, a saturation of from about 0 to about 10%, and a value of from about 50% to about 100%.

In some embodiments, the colored coverlay substrate 220 may be used in assays using fluorescence-based measurements. Assays using fluorescence-based measurements may utilize fluorescent light in detection of a target compound. For assays using fluorescence-based measurements, the color of the colored coverlay substrate 220 may comprise a dark color such as dark gray, near-black, black, or any other dark color. For example, the color of the colored coverlay substrate 220 may comprise a HSV comprising a hue of from about 0 to about 80, a saturation of from about 0 to about 25% and/or a value of from about 0% to about 50%.

In some embodiments, the top plate 210 of the DMF cartridge 200 may comprise PCB substrate, a glass substrate, a silicon substrate, a polyimide substrate, or any other known material used in existing DMF cartridges. In some embodiments, the top plate 210 is transparent or substantially transparent. In some embodiments, the top plate 210 is opaque or substantially opaque.

In some embodiments, the bottom plate 215 of the DMF cartridge 200 may comprise a PCB substrate, a glass substrate, a silicon substrate, a polyimide substrate, or any other known material used in existing DMF cartridges. In some embodiments, the bottom plate 215 is transparent or substantially transparent. In some embodiments, the bottom plate 215 is opaque or substantially opaque. In some embodiments, the bottom plate comprises a colored coverlay substrate 220.

In some embodiments, color may be provided to the colored coverlay substrate 220 of the bottom plate 215 by existing methods. For example, a color may be provided to the colored coverlay substrate 220 (e.g., the PCB substrate, glass substrate, or silicon substrate) of the bottom plate 215 through the addition of a colored pigment during the manufacture of the colored coverlay substrate 220 or through an application of a color coating on the colored coverlay substrate 220. Other means for providing a color to the colored coverlay substrate 220 may include. In another embodiment, a colored film or tape can be added to the bottom plate 215. The colored film or tape may comprise any of the colors described herein. For example, a white polyimide film or tape can be applied or added to the bottom plate 215. As another example, a black film or tape may be applied or added to the bottom plate 215.

In other embodiments, the top plate 210 may comprise a material that is transparent to light. For example, the top plate 210 can be selected from a transparent material, such as quartz, cyclo olefin polymer (COP), Cyclic olefin copolymer (COC), ceramics, or any other materials that is transparent or substantially transparent to light. The material used for the top plate 210 and/or the color of colored coverlay substrate 220 can be selected based on the anticipated assay that may be performed on the DMF cartridge 200 and on the anticipated target analyte to be detected (e.g., based on the wavelength desired for interrogation of a given target analyte). For example, in some embodiments, the color of the colored coverlay substrate 220 can be selected and/or tuned to maximize the dynamic range and/or linear range of detection for one or more reporter moieties. In other embodiments, the color of the colored coverlay substrate 220 can be selected and/or tuned to maximize the limit of detection (LOD) for one or more reporter moieties. In another embodiment, the color of the colored coverlay substrate 220 can be selected and/or tuned to maximize the assay sensitivity.

In some embodiments, the DMF cartridge 200 may comprise a gap (containing a droplet therein) 240 that is formed by the separation or spacing between the top plate 210 and the bottom plate 215 . . . . As noted above, the bottom plate 215 may include a plurality of electrodes 230 for droplet manipulation. In another embodiment, the DMF cartridge 200 can be patterned with an actuation grid (not shown) for droplet manipulation. In some embodiments, the DMF cartridge may comprise a sample inlet port (not shown), which may be in fluid communication with the plurality of droplet operation electrodes 230 on the colored coverlay substrate 220, and which may be operable to receive a fluid sample to be analyzed for the presence of a target analyte (e.g., from a syringe).

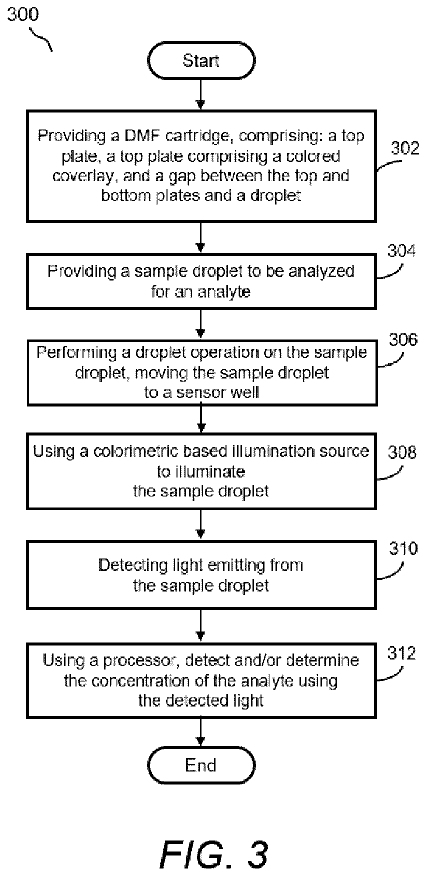

FIG. 3 illustrates a method 300 for analyzing an analyte of interest in a droplet using an optical detection operation, in accordance with one aspect of the inventive concepts described herein. As shown in FIG. 3, at step 302, the method may utilize a DMF cartridge that may include a colored coverlay substrate to provide for enhanced or improved detection of a target analyte in a fluid sample.

At step 302, a DMF cartridge may be provided. In general, any DMF cartridge that may include a colored coverlay substrate can be used in the practice of the inventive concepts described herein. More specifically, a DMF cartridge comprising a bottom plate with a colored coverlay substrate can be used, such as disclosed herein in conjunction with FIG. 2. As exemplified in method 300, at step 302, the DMF cartridge may comprises: (i) a top plate, wherein the top plate comprises a top plate substrate; (ii) a bottom plate, wherein the bottom plate comprises a colored coverlay substrate having a plurality of electrodes operable to perform droplet operations; and (iii) a droplet operation gap between the top plate and the bottom plate.

At step 304, a sample droplet to be analyzed for the presence of an analyte of interest may be provided. In general, the cartridges and methods of the present inventive concepts described herein can be used for detection and analysis of a wide array of possible target analytes. For example, the target analyte may include, but is not limited to, small molecules, proteins, peptides, antibodies, lipids, cells, nucleic acids, atoms, ions, or any combination thereof. Likewise, a wide array of possible biological and/or clinical sample types can be used in the practice of the inventive concepts. Non-limiting examples include blood, plasma, serum, tears, saliva, urine, or any combination thereof. In some cases, the clinical sample may include a dried blood sample. In some cases, the clinical sample may include a fresh blood sample.

In some embodiments, the DMF cartridge may include a plurality of electrodes for droplet operation, which may be controlled by a controller or a processor for droplet manipulation within the droplet operations gap of the DMF cartridge. At step 306, a droplet operation may be performed on the droplet sample, thereby moving the sample droplet to a sensor well in the DMF cartridge for analysis at steps 308 through 312.

At step 308, an optical detection operation may be performed by using an illumination source to illuminate the sample droplet in the sensor well. In some embodiments, the optical detection operation selected may depend on the target analyte being analyzed. For example, the optical detection operation can be selected from one or more of: absorbance-based optical detection operations, colorimetric-based optical detection operations, luminescence-based optical detection operations, fluorescence-based optical detection operations, or any combination thereof. In some embodiments, a wide variety of illumination sources can be used depending on the type of detection operation being performed. When using absorbance-based, colorimetric-based, or luminescence-based optical detection, a white light emitting diode (LED) can be used. When using fluorescence-based optical detection, the selection of an illumination source may depend on the excitation wavelength needed for detection of the fluorescent reporter moiety used. For operation below the visible range (e.g., 100-400 nm) of the spectrum, an ultraviolet (UV) light source such as a UV LED may be used. For operation above the visible range (e.g., 800-2500 nm), an infrared (IR) source such as an IR LED may be used.

At step 310, light emitting from the sample droplet may be detected using an optical measuring device. The optical measuring device may comprise a charge coupled device, a photodetector, a spectrometer, a photodiode array, or any combination thereof, to obtain light intensity readings. In some embodiments, an appropriate measuring device may be selected based on the illumination source used.

In some embodiments, light can be detected and quantified in the Cyan, Magenta, Yellow, and Key (CMYK) color space. For example, light detected in the CMYK color space can be used to quantify reporter moieties that demonstrate a correlation between reporter moiety concentration and intensity of color, specifically when those colors are cyan, magenta, and/or yellow. In one embodiment, 3′,5,5′-Tetramethylbenzidine (e.g., TMB) can be used as a reporter moiety (e.g., in an ELISA assay). As TMB is oxidized (e.g., to TMB⁺), it may become a more intense blue as the concentration of TMB⁺ is increased, which can be detected and quantified using light in the CMYK color space. In the CMYK model, white may be the natural color of the background and the measured color values (e.g., cyan, magenta, yellow and key) of a white background may be about 0, 0, 0, 0. As the concentration of oxidized TMB (TMB⁺) increases, and the color becomes more intense, the cyan value may increase and other values (e.g., magenta, yellow, and key) may also increase. As such, in the practice of this embodiment, to maximize the detection limit of a substrate like TMB⁺ on the digital microfluidics platform (DMF), a white coverlay dielectric can be used for imaging purposes and the color values may be detected and measured in CMYK mode.

In another embodiment, light can be detected and quantified in the HSV color space. For example, light detected in the HSV color space can be used to quantify reporter moieties that demonstrate a correlation between reporter moiety concentration and perceived color/hue (such as red, yellow, blue, etc.). In one embodiment, gold, silver, platinum, palladium, copper, gold coated silver, gold coated or silver coated nanoparticles, or any combination thereof, can be used as a reporter moiety.

In another embodiment, gold nanourchins can be used as a reporter moiety (e.g., in a pELISA assay). For example, against a white colored coverlay in a pELISA assay, the perceived color/hue of gold nanourchins (of ~70 nm in diameter) on a white background may change from blue (~220-230 hue degrees), to violet (~270 degrees), to magenta (~300 degrees), and to pink (~340 degrees) as the shape of the nanoparticles is modified by etching of oxidized TMB. However, the perceived color/hue (and change in color/hue) of gold nanourchins may be less dynamic against an orange colored coverlay. As such, in the practice of this embodiment, to maximize the detection limit of gold nanourchins as a reporter moiety on the digital microfluidics platform (DMF), a white coverlay dielectric can be used for imaging purposes and the color values may be detected and measured in HSV mode. In some embodiments, for applications on DMF where it may be necessary to quantify and/or measure the change in color/hue of reporter moieties, the selection of a colored coverlay dielectric should consider the impact on the dynamic (and/or linear) range of detection, the LOD, and the assay sensitivity.

In some embodiments, the optical measuring device may comprise a combination of two or more types of detectors for detection of multiple reporter moieties. For example, multiple detection modes can be used (e.g., HSV, CMYK, etc.) to maximize the overall assay dynamic range without compromising the LOD in a pELISA to measure color/hue and/or change in color/hue as gold nanourchins are etched by oxidized TMB and to measure CMYK values of oxidized TMB.

Finally, and as shown in FIG. 3, at step 312, a processor can be used to analyze the detected light and to determine whether or not a target analyte is present. In other applications, the processor can be used to analyze the detected light, or the intensity of the detected light to determine the concentration of the target analyte. In some embodiments, the detected light, or the intensity of light detected, can be used to detect the presence of a reporter moiety (e.g., in an ELISA assay), and thereby detect the presence of an analyte and/or determine the concentration of an analyte in the sample.

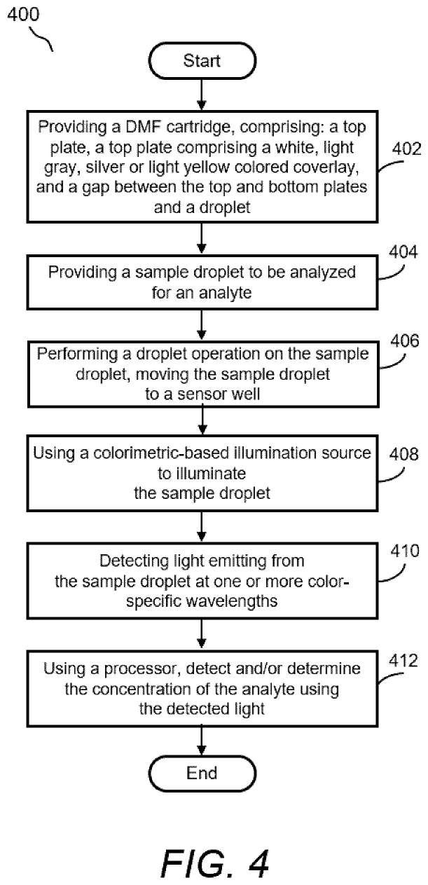

FIG. 4 illustrates a method 400 for analyzing an analyte of interest in a droplet using a colorimetric-based optical detection operation, in accordance with one aspect of the inventive concepts described herein. As shown in FIG. 4, at step 402, the method may utilize a DMF cartridge that may include a white colored coverlay substrate that may provide for enhanced or improved detection of a target analyte in a fluid sample.

At step 402, a DMF cartridge may be provided. In general, any DMF cartridge that includes a colored coverlay substrate can be used in the practice of the present inventive concepts described herein. More specifically, a DMF cartridge comprising a bottom plate with a colored coverlay substrate can be used, such as disclosed herein in conjunction with FIG. 2. As exemplified in method 300, at step 302, the DMF cartridge may comprises: (i) a top plate, wherein the top plate comprises a top plate substrate; (ii) a bottom plate, wherein the bottom plate comprises a white, light gray, silver, or light yellow colored coverlay substrate and a plurality of electrodes operable to perform droplet operations; and (iii) a droplet operation gap between the top plate and the bottom plate.

At step 404, a sample droplet to be analyzed for the presence of an analyte of interest may be provided. In general, the cartridges and methods of the present inventive concepts described herein can be used for detection and analysis of a wide array of possible target analytes. For example, the target analyte may include, but are not limited to, small molecules, proteins, peptides, antibodies, lipids, cells, nucleic acids, atoms, ions, or any combination thereof. Likewise, a wide array of possible biological and/or clinical sample types can be used in the practice of the inventive concepts described herein. Non-limiting examples include blood, plasma, serum, tears, saliva, urine, or any combination thereof. In some cases, the clinical sample may include a dried blood sample. In some cases, the clinical sample may include a fresh blood sample.

In some embodiments, the DMF cartridge may include a plurality of electrodes for droplet operations, which may be controlled by a controller or a processor for droplet manipulation within the droplet operations gap of the DMF cartridge. At step 406, a droplet operation may be performed on the droplet sample, thereby moving the sample droplet to a sensor well in the DMF cartridge for analysis at steps 408 through 412.

At step 408, a colorimetric-based optical detection operation may be performed by using an illumination source to illuminate the sample droplet in the sensor well. In accordance with this embodiment, the optical detection operation is a colorimetric-based one. A wide variety of illumination sources can be used depending on the type of detection operation being performed. In some embodiments, a white light emitting diode (LED) is used.

At step 410, light emitting from the sample droplet may be detected using an optical measuring device. The optical measuring device may comprise a charge coupled device, a photodetector, a spectrometer, a photodiode array, or any combination thereof, to obtain light intensity readings. In one embodiment, light can be detected and quantified in the CMYK color space and/or in the HSV color space, as previously described in conjunction with FIG. 3. For example, light detected in the CMYK color space (e.g., cyan, magenta, yellow and key) can be used to quantify reporter moieties (e.g., TMB) that may demonstrate a correlation between reporter moiety concentration and intensity of color against the white colored coverlay (e.g., in an ELISA assay). In the practice of this embodiment, a white coverlay dielectric may be used to maximize the detection limit of a substrate like TMB⁺ on the digital microfluidics platform (DMF). In another embodiment, light can be detected and quantified in the HSV color space to detect and quantify reporter moieties (e.g., etching of gold nanourchins) that may demonstrate a correlation between reporter moiety concentration and perceived color/hue (e.g., such as red, yellow, blue, etc.) against the white colored coverlay (e.g., in a pELISA assay). In the practice of this embodiment, a white coverlay dielectric may be used to maximize the detection limit of gold nanourchins as a reporter moiety on the digital microfluidics platform (DMF).

In other embodiments, the optical measuring device may comprise a combination of two or more types of detectors for detection of multiple reporter moieties. For example, multiple detection modes can be used (e.g., HSV, CMYK, etc.) to maximize the overall assay dynamic range without compromising the LOD in a pELISA to measure color/hue and/or change in color/hue as gold nanourchins are etched by oxidized TMB and to measure CMYK values of oxidized TMB.

Finally, as shown in FIG. 4, at step 412, a processor can be used to analyze the detected light and to determine whether or not a target analyte is present. In other applications, the processor can be used to analyze the detected light, or the intensity of the detected light to determine the concentration of the target analyte. In some embodiments, the detected light, or the intensity of light detected, can be used to detect the presence of a reporter moiety (e.g., in an ELISA assay), and thereby detect the presence of an analyte and/or determine the concentration of an analyte in the sample.

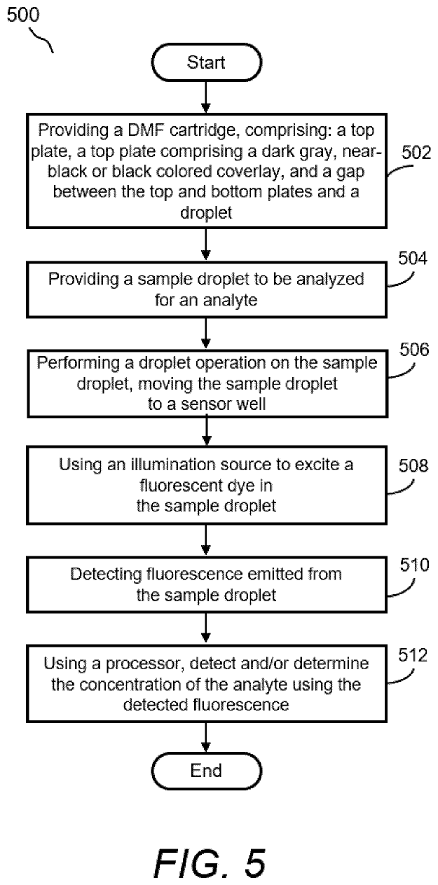

FIG. 5 illustrates a method 500 for analyzing an analyte of interest in a droplet using fluorescence-based optical detection operation, in accordance with one aspect of the inventive concepts described herein. As shown in FIG. 5, at step 502, the method may utilize a DMF cartridge that includes a black colored coverlay substrate to provide for enhanced or improved detection of a target analyte in a fluid sample.

At step 502, a DMF cartridge may be provided. In general, any DMF cartridge that includes a dark gray, near-black or black colored coverlay substrate can be used in the practice of the present inventive concepts described herein. In some embodiments, the use of a black coverlay substrate can minimize scattered and reflected light in fluorescence-based applications. More specifically, a DMF cartridge comprising a bottom plate with a black colored coverlay substrate can be used, such as disclosed herein in conjunction with FIG. 2. As exemplified in method 500, at step 502, the DMF cartridge may comprises: (i) a top plate, wherein the top plate comprises a top plate substrate; (ii) a bottom plate, wherein the bottom plate comprises a dark gray, near-black or black colored coverlay substrate and a plurality of electrodes operable to perform droplet operations; and (iii) a droplet operation gap between the top plate and the bottom plate.

At step 504, a sample droplet to be analyzed for the presence of an analyte of interest may be provided. In general, the cartridges and methods of the present inventive concepts described herein can be used for detection and analysis of a wide array of possible target analytes. For example, the target analyte may include, but are not limited to, small molecules, proteins, peptides, antibodies, lipids, cells, nucleic acids, atoms, ions, or any combination thereof. Likewise, a wide array of possible biological and/or clinical sample types may be used in the practice of the inventive concepts. Non-limiting examples include blood, plasma, serum, tears, saliva, and urine. In some cases, the clinical sample may include a dried blood sample. In some cases, the clinical sample may include a fresh blood sample.

In some embodiments, the DMF cartridge may include a plurality of electrodes for droplet operation, which may be controlled by a controller or a processor for droplet manipulation within the droplet operations gap of the DMF cartridge. At step 406, a droplet operation may be performed on the droplet sample, thereby moving the sample droplet to a sensor well in the DMF cartridge for analysis at steps 508 through 512.

At step 508, a fluorescence-based detection operation may be performed by using an illumination source capable of exciting a fluorescent reporter moiety (e.g., a fluorescent dye) contained in the sample droplet and used for detection and quantification of a target analyte. A wide variety of illumination sources can be used depending on the detection operation being performed. The selection of an illumination source may depend on the excitation wavelength needed for detection of the fluorescent reporter moiety used. When the excitation wavelength needed for detection is in the visible range, a white light emitting diode (LED) can be used. For operation below the visible range (100-400 nm) of the spectrum, an ultraviolet (UV) light source such as a UV LED may be used. For operation above the visible range (800-2500 nm) an infrared (IR) source such as an IR LED may be used.

At step 510, light emitting from the sample droplet may be detected using an optical measuring device. The optical measuring device may comprise a charge coupled device, a photodetector, a spectrometer, a photodiode array, or any combination thereof, to obtain light intensity readings. An appropriate measuring device may be selected based on the illumination source used.

Finally, at step 512, a processor can be used to analyze the detected light and to determine whether or not a target analyte is present. In other applications, the processor can be used to analyze the detected light, or the intensity of the detected light to determine the concentration of the target analyte. In some embodiments, the detected light, or the intensity of light detected, can be used to detect the presence of a reporter moiety (e.g., in an ELISA assay), and thereby detect the presence of an analyte and/or determine the concentration of an analyte in the sample.

### Terms and Definitions

Unless otherwise defined, all technical terms used herein have the same meaning as commonly understood by one of ordinary skill in the art to which this disclosure belongs.

“Droplet Actuator” means a device for manipulating droplets. Microfluidics devices, microfluidics cartridges, digital microfluidics (DMF) devices, and DMF cartridges are examples of droplet actuators. Certain droplet actuators will include one or more substrates arranged with a droplet operations gap therebetween and electrodes associated with (e.g., patterned on, layered on, attached to, and/or embedded in) the one or more substrates and arranged to conduct one or more droplet operations. For example, certain droplet actuators will include a base (or bottom) substrate, droplet operations electrodes associated with the substrate, one or more dielectric layers atop the substrate and/or electrodes, and optionally one or more hydrophobic layers atop the substrate, dielectric layers and/or the electrodes forming a droplet operations surface. A top substrate may also be provided, which is separated from the droplet operations surface by a gap, commonly referred to as a droplet operations gap. Droplet actuators will include various electrode arrangements on the top and/or bottom substrates. During droplet operations it is preferred that droplets remain in continuous contact or frequent contact with a ground or reference electrode. A ground or reference electrode may be associated with the top substrate facing the gap, the bottom substrate facing the gap, or within the gap itself. Where electrodes are provided on both substrates, electrical contacts for coupling the electrodes to a droplet actuator instrument for controlling or monitoring the electrodes may be associated with one or both plates. In some cases, electrodes on one substrate are electrically coupled to the other substrate so that only one substrate is in contact with the droplet actuator. Where multiple substrates are used, a spacer may be provided between the substrates to determine the height of the gap therebetween and define on-actuator dispensing reservoirs. The spacer height may, for example, be from about 5 μm to about 1000 μm, or about 100 μm to about 400 μm, or about 200 μm to about 350 μm, or about 250 μm to about 300 μm, or about 275 μm. The spacer may, for example, be formed of features or layers projecting from the top or bottom substrates, and/or a material inserted between the top and bottom substrates. One or more openings may be provided in the one or more substrates for forming a fluid path through which liquid may be delivered into the droplet operations gap.

In some cases, the top and/or bottom substrate of a droplet actuator includes a PCB substrate that is coated with a dielectric, such as a polyimide dielectric, which may in some cases also be coated or otherwise treated to make the droplet operations surface hydrophobic. Various materials are also suitable for use as the dielectric component of the droplet actuator. In some cases, the top and/or bottom substrate of a droplet actuator includes a glass or silicon substrate on which features have been patterned using process technology borrowed from semiconductor device fabrication including the deposition and etching of thin layers of materials using microlithography. The top and/or bottom substrate may consist of a semiconductor backplane (e.g., a thin-film transistor (TFT) active-matrix controller) on which droplet operations electrodes have been formed.

Electrodes of a droplet actuator are typically controlled by a controller or a processor, which is itself provided as part of a system, which may include processing functions as well as data and software storage and input and output capabilities. Reagents may be provided on the droplet actuator in the droplet operations gap or in a reservoir fluidly coupled to the droplet operations gap. The reagents may be in liquid form, e.g., droplets, or they may be provided in a reconstitutable form in the droplet operations gap or in a reservoir fluidly coupled to the droplet operations gap. Reconstitutable reagents may typically be combined with liquids for reconstitution.

“Droplet operation” means any manipulation of a droplet on a droplet actuator. A droplet operation may, for example, include: loading a droplet into the droplet actuator; dispensing one or more droplets from a source droplet; splitting, separating or dividing a droplet into two or more droplets; transporting a droplet from one location to another in any direction; merging or combining two or more droplets into a single droplet; diluting a droplet; mixing a droplet; agitating a droplet; deforming a droplet; retaining a droplet in position; incubating a droplet; heating a droplet; vaporizing a droplet; cooling a droplet; disposing of a droplet; transporting a droplet out of a droplet actuator; other droplet operations described herein; and/or any combination of the foregoing. The terms “merge,” “merging,” “combine,” “combining” and the like are used to describe the creation of one droplet from two or more droplets. It should be understood that when such a term is used in reference to two or more droplets, any combination of droplet operations that are sufficient to result in the combination of the two or more droplets into one droplet may be used. For example, “merging droplet A with droplet B,” can be achieved by transporting droplet A into contact with a stationary droplet B, transporting droplet B into contact with a stationary droplet A, or transporting droplets A and B into contact with each other. The terms “splitting,” “separating” and “dividing” are not intended to imply any particular outcome with respect to volume of the resulting droplets (e.g., the volume of the resulting droplets can be the same or different) or number of resulting droplets (the number of resulting droplets may be 2, 3, 4, 5 or more). The term “mixing” refers to droplet operations which result in more homogenous distribution of one or more components within a droplet. Examples of “loading” droplet operations include microdialysis loading, pressure assisted loading, robotic loading, passive loading, and pipette loading. Droplet operations may be electrode mediated. In some cases, droplet operations are further facilitated by the use of hydrophilic and/or hydrophobic regions on surfaces and/or by physical obstacles. For examples of droplet operations, see the patents and patent applications cited above under the definition of “droplet actuator.” Impedance and/or capacitance sensing and/or imaging techniques may sometimes be used to determine or confirm the outcome of a droplet operation. Generally speaking, the sensing or imaging techniques may be used to confirm the presence or absence of a droplet at a specific electrode. For example, the presence of a dispensed droplet at the destination electrode following a droplet dispensing operation confirms that the droplet dispensing operation was effective. Similarly, the presence of a droplet at a detection spot at an appropriate step in an assay protocol may confirm that a previous set of droplet operations has successfully produced a droplet for detection. Droplet transport time can be quite fast. For example, in various embodiments, transport of a droplet from one electrode to the next may be completed within about 1 sec, or about 0.1 sec, or about 0.01 sec, or about 0.001 sec. In one embodiment, the electrode is operated in AC mode but is switched to DC mode for imaging. It is helpful for conducting droplet operations for the footprint area of droplet to be similar to or larger than the electrowetting area; in other words, 1×-, 2×- 3×-droplets are usefully controlled and/or operated using 1, 2, and 3 electrodes, respectively. If the droplet footprint is greater than number of electrodes available for conducting a droplet operation at a given time, the difference between the droplet size and the number of electrodes should typically not be greater than 1; in other words, a 2× droplet is usefully controlled using 1 electrode and a 3× droplet is usefully controlled using 2 electrodes. When droplets include beads, it is useful for droplet size to be equal to the number of electrodes controlling the droplet, e.g., transporting the droplet.

“Filler fluid” means a fluid associated with a droplet operations substrate of a droplet actuator, which fluid is sufficiently immiscible with a droplet phase to render the droplet phase subject to electrode-mediated droplet operations. For example, the droplet operations gap of a droplet actuator is typically filled with a filler fluid. The filler fluid may, for example, be or include a low-viscosity oil, such as silicone oil or hexadecane. The filler fluid may be or include a halogenated oil, such as a fluorinated or perfluorinated oil. The filler fluid may fill the entire gap of the droplet actuator or may only coat one or more surfaces of the droplet actuator. Filler fluids may be selected to improve droplet operations and/or reduce loss of reagent or target substances from droplets, reduce formation of unwanted microdroplets, reduce cross contamination between droplets, reduce contamination of droplet actuator surfaces, reduce degradation of droplet actuator materials, reduce evaporation of droplets, etc. For example, filler fluids may be selected for compatibility with droplet actuator materials. As an example, fluorinated filler fluids may be usefully employed with fluorinated surface coatings. Fluorinated filler fluids are useful to reduce loss of lipophilic compounds, such as umbelliferone substrates like 6-hexadecanoylamino-4-methylumbelliferone substrates (e.g., for use in Krabbe, Niemann-Pick, or other assays); Filler fluids may, for example, be doped with surfactants or other additives. For example, additives may be selected to improve droplet operations and/or reduce loss of reagent or target substances from droplets, formation of microdroplets, cross contamination between droplets, contamination of droplet actuator surfaces, degradation of droplet actuator materials, etc. Composition of the filler fluid, including surfactant doping, may be selected for performance with reagents or samples used in the specific assay protocols and effective interaction or non-interaction with droplet actuator materials. For example, fluorinated oils may in some cases be doped with fluorinated surfactants, e.g., Zonyl FSO-100 (Sigma-Aldrich) and/or others.

The terms “top,” “bottom,” “over,” “under,” and “on” are used throughout the description with reference to the relative positions of components of the droplet actuator, such as relative positions of top and bottom substrates of the droplet actuator. It will be appreciated that in many cases the droplet actuator is functional regardless of its orientation in space.

When a liquid in any form (e.g., a droplet or a continuous body, whether moving or stationary) is described as being “on”, “at”, or “over” an electrode, array, matrix or surface, such liquid may be either in direct contact with the electrode/array/matrix/surface, or may be in contact with one or more layers or films that are interposed between the liquid and the electrode/array/matrix/surface. In one example, filler fluid can be considered as a dynamic film between such liquid and the electrode/array/matrix/surface.

When a droplet is described as being “on” or “loaded on” a droplet actuator, it should be understood that the droplet is arranged on the droplet actuator in a manner which facilitates using the droplet actuator to conduct one or more droplet operations on the droplet, the droplet is arranged on the droplet actuator in a manner which facilitates sensing of a property of or a signal from the droplet, and/or the droplet has been subjected to a droplet operation on the droplet actuator.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” “comprising,” “include,” “includes,” and “including,” are intended to be non-limiting, such that recitation of items in a list is not to the exclusion of other like items that may be substituted or added to the listed items

Terms like “preferably,” “commonly,” and “typically” are not utilized herein to limit the scope of the claimed embodiments or to imply that certain features are critical or essential to the structure or function of the claimed embodiments. These terms are intended to highlight alternative or additional features that may or may not be utilized in a particular embodiment of the present disclosure.

The term “substantially” is utilized herein to represent the inherent degree of uncertainty that may be attributed to any quantitative comparison, value, measurement, or other representation and to represent the degree by which a quantitative representation may vary from a stated reference without resulting in a change in the basic function of the subject matter at issue.

Various modifications and variations of the disclosed methods, compositions, and uses of the inventive concepts will be apparent to the skilled person without departing from the scope and spirit of the inventive concepts. Although the inventive concepts have been disclosed in connection with specific preferred aspects or embodiments, the inventive concepts as claimed should not be unduly limited to such specific aspects or embodiments.

The present inventive concepts may be implemented using hardware, software, or a combination thereof and may be implemented in one or more computer systems or other processing systems. In one aspect, the inventive concepts are directed toward one or more computer systems capable of carrying out the methods described herein. Software may be stored in a computer-readable non-transitory storage medium.

Although the foregoing subject matter has been described in some detail by way of illustration and example for purposes of clarity of understanding, it will be understood by those skilled in the art that certain changes and modifications can be practiced within the scope of the appended claims

As used herein, the term “about” in some cases refers to an amount that is approximately the stated amount.

As used herein, the term “about” refers to an amount that is near the stated amount by 10%, 5%, or 1%, including increments therein.

As used herein, the term “about” in reference to a percentage refers to an amount that is greater or less the stated percentage by 10%, 5%, or 1%, including increments therein.

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

### EXAMPLES

The following illustrative examples are representative of embodiments of the software applications, systems, and methods described herein and are not meant to be limiting in any way.

### Example 1. Cyan Counts (in CMYK Color Model) of Various Concentrations of TMB⁺ on White Coverlay Dielectric

Various concentrations of oxidized 3,3′,5,5′-Tetramethylbenzidine (TMB⁺) droplets (e.g., from 0 μM to 24 μM TMB⁺) were dispensed onto a white coverlay dielectric substrate of a DMF cartridge. The concentrations of TMB⁺ in each of the droplets were quantified using the Cyan, Magenta, Yellow, and Key (CMYK) color model (specifically by measuring the cyan counts).

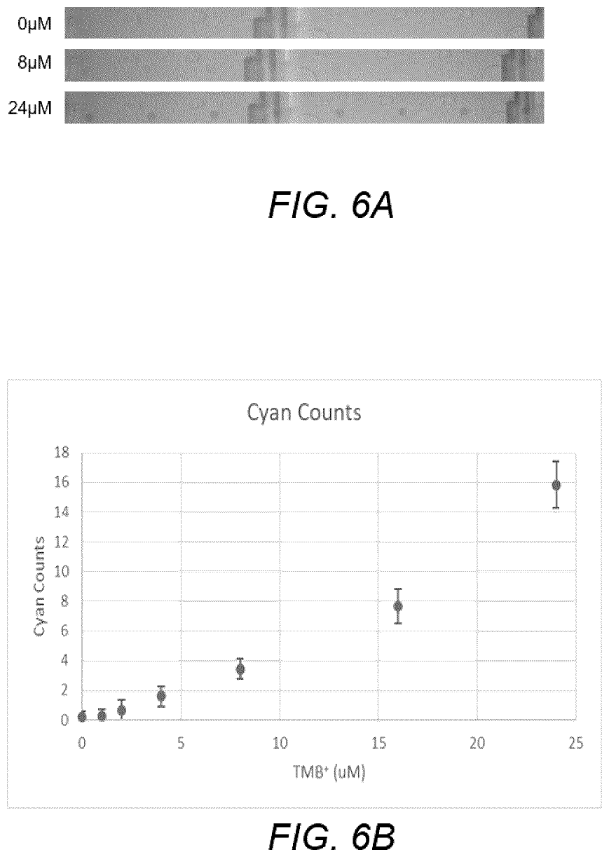

Results are shown in FIGS. 6A-6B. FIG. 6A shows images of 0 μM TMB⁺ (e.g., non-oxidized TMB), 8 μM TMB⁺, and 24 μM TMB⁺ on the white coverlay dielectric. As shown in FIG. 6A, the color tonality of the various concentrations TMB⁺ (e.g., 0 μM, 8 μM, and 24 μM) was distinguishable against the white coverlay dielectric. FIG. 6B shows a graph depicting the cyan counts detected at various concentrations of oxidized TMB, from 0 μM TMB⁺ (e.g., non-oxidized TMB) to 24 μM TMB⁺, on the white coverlay dielectric. As shown in FIG. 6B, a larger number of cyan counts were observed in droplets comprising larger concentrations of TMB⁺. The limit of detection for TMB⁺ using this method was determined to be around 4 μM.

### Example 2. Etched & Unetched Gold Nanourchins (AuNUs) on Orange and White Coverlay Dielectrics

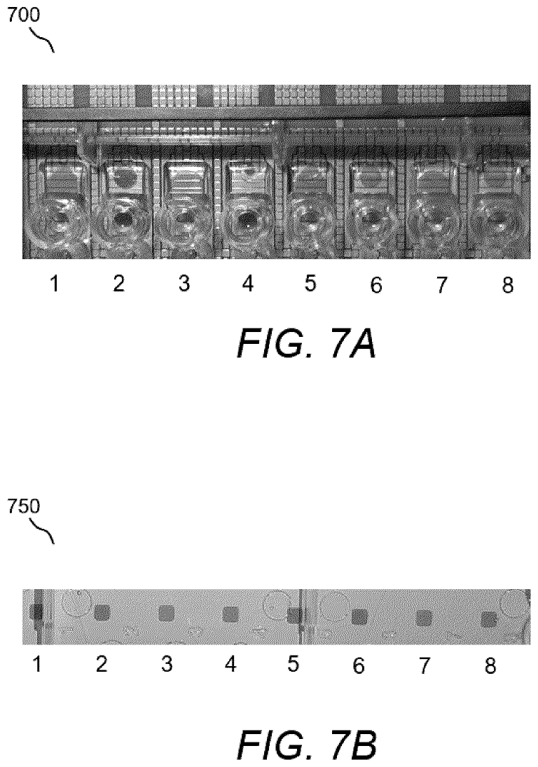

As shown in FIGS. 7A-7B, various concentrations of etched (using TMB⁺) and unetched gold nanourchin (AuNUs) droplets (from 3 OD to 7.5 OD) were dispensed onto either an orange coverlay dielectric (as shown in FIG. 7A) or a white coverlay dielectric substrate (as shown in FIG. 7B). Color values were measured using the HSV color space.

Results are shown in FIGS. 7A-7B. FIG. 7A shows the following from left to right: 1, etched AuNUs (8 OD); 2. unetched AuNUs (8 OD); 3, etched AuNUs (6 OD); 4. unetched AuNUs (6 OD); 5, etched AuNUs (4.3 OD); 6. unetched AuNUs (4.3 OD); 7, etched AuNUs (3 OD); and 8. unetched AuNUs (3 OD). FIG. 7B shows the following from left to right: 1. unetched AuNUs (7.5 OD); 2, etched AuNUs (7.5 OD); 3, etched AuNUs (6 OD); 4. unetched AuNUs (6 OD); 5. unetched AuNUs (7.5 OD); 6, etched AuNUs (7.5 OD); 7, etched AuNUs (6 OD); and 8. unetched AuNUs (6 OD).

As shown in FIGS. 7A-7B, the selection of the colored coverlay affects the perceived color of plasmonic nanoparticles (e.g., such as gold nanourchins). As shown in FIG. 7A, unetched gold nanourchins and gold nanourchins that have been etched (by TMB⁺) appeared as different shades of orange-brown against an orange coverlay dielectric. However, as shown in FIG. 7B, against a white coverlay dielectric, etched gold nanourchins appeared pink and unetched nanourchins appeared blue.

When measuring the color values of etched and unetched gold nanourchins in the HSV color space, there was minimal change in the hue value of etched and unetched gold nanourchins against an orange coverlay dielectric. However, against a white coverlay dielectric, the change in hue was observed to be much larger. Based on these observations, where it is necessary to monitor or measure the change in hue of gold nanourchins, a white coverlay dielectric may have an advantage of providing a greater dynamic range in measured hue value (as opposed to using an alternate coverlay dielectric, such as an orange coverlay dielectric).

TABLE 1
Hue Measurements of Etched & Unetched Gold
Nanourchins on White Coverlay Dielectric

| | Unetched AuNUs | | Etched AuNUs | |
|---|---|---|---|---|
| AuNUs Concentration | Position 1 | Position 5 | Position 2 | Position 6 |
| 7.5 OD | 223 | 229 | 326 | 323 |
| | Average Hue = 226 | | Average Hue = 324.5 | |
| | Position 4 | Position 8 | Position 3 | Position 7 |
| 6.0 OD | 224 | 218 | 328 | 339 |

| | |
|---|---|
| Average Hue = 221 | Average Hue = 333.5 |

### Example 3. Change in AuNUs Hue Vs Time

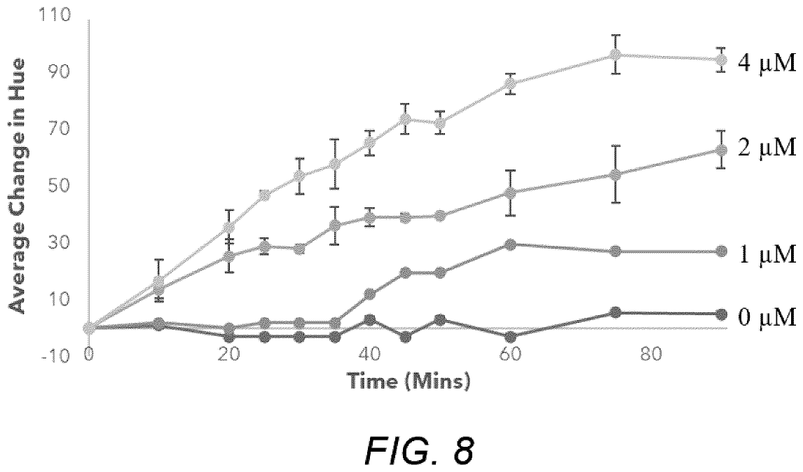

The change in Hue of gold nanourchins on a white coverlay dielectric was measured at various times in the presence of oxidized TMB (from a concentration of 0 μM to 4 μM TMB⁺) and plotted on a graph as shown in in FIG. 8. FIG. 8 shows the following: 0 μM: Consists of 3 droplet units of gold nanourchins mixed with 1 droplet unit of non-oxidized TMB; 1 μM: Consists of 3 droplet units of gold nanourchins mixed with 1 droplet unit of 1 μM TMB⁺; 2 μM consist of 3 droplet units of gold nanourchins mixed with 1 droplet unit of 2 μM TMB⁺; and 4 μM: Consists of 3 droplet units of gold nanourchins mixed with 1 droplet unit of 4 μM TMB⁺.

When gold nanourchins were used for signal amplification by measuring the change in hue of the gold nanourchins against a white coverlay dielectric in the HSV color space, the limit of detection for TMB⁺ was observed to be lower than 1 μM (with even 1 μM TMB⁺ being distinguishable from 0 μM at 40 min), as shown in FIG. 8. However, these observations show that it may be difficult to distinguish between higher concentrations of TMB⁺ (e.g., >4 μM) using gold nanourchins for signal amplification because the dynamic/linear range of detection was observed to be lower than that of measuring TMB⁺ in the CMYK mode against a white coverlay dielectric. Therefore, the advantages of both detection modes may be utilized by combining both detection modes in a pELISA.

While preferred embodiments of the present disclosure have been shown and described herein, it will be obvious to those skilled in the art that such embodiments are provided by way of example only. It is not intended that the disclosure be limited by the specific examples provided within the specification. While the disclosure has been described with reference to the aforementioned specification, the descriptions and illustrations of the embodiments herein are not meant to be construed in a limiting sense. Numerous variations, changes, and substitutions will now occur to those skilled in the art without departing from the disclosure. Furthermore, it shall be understood that all aspects of the disclosure are not limited to the specific depictions, configurations or relative proportions set forth herein which depend upon a variety of conditions and variables. It should be understood that various alternatives to the embodiments of the disclosure described herein may be employed in practicing the disclosure. It is therefore contemplated that the inventive concepts shall also cover any such alternatives, modifications, variations or equivalents. It is intended that the following claims define the scope of the disclosure and that methods and structures within the scope of these claims and their equivalents be covered thereby.

1. A digital microfluidics (DMF) cartridge, the DMF cartridge comprising:
a top plate, wherein the top plate comprises a top plate substrate;
a bottom plate, wherein the bottom plate comprises a bottom plate substrate and a colored coverlay substrate; and
a droplet operation gap between the top plate and the bottom plate.

2. The DMF cartridge of claim 1, wherein the bottom plate substrate comprises a printed circuit board (PCB) substrate, a glass substrate, a silicon substrate, or a polyamide substrate, and wherein the PCB substrate, the glass substrate, the silicon substrate, or the polyimide substrate further comprises the colored coverlay substrate.

3. The DMF cartridge of claim 1, wherein the top plate substrate comprises a printed circuit board (PCB) substrate, a glass substrate, a silicon substrate, or a polyamide substrate, and wherein the PCB substrate, the glass substrate, the silicon substrate, or the polyimide substrate further comprises the colored coverlay substrate.

4. The DMF cartridge of claim 1, wherein the color of the colored coverlay substrate is white, light gray, silver, orange, light yellow, dark gray, near-black or black.

5. The DMF cartridge of claim 1, wherein the color of the colored coverlay substrate is white, light gray, silver, or light yellow.

6. The DMF cartridge of claim 5, wherein the white, light gray, silver, light yellow color of the colored coverlay substrate comprises a Hue, Saturation, and Value (HSV).

7. The DMF cartridge of claim 6, wherein the HSV comprises a hue of from about 0 to about 70, a saturation of from about 0 to about 10%, and/or a value of from about 50% to about 100%.

8. The DMF cartridge of claim 4, wherein the color of the colored coverlay substrate is black.

9. The DMF cartridge of claim 4, wherein the dark gray, near-black or black color of the colored coverlay substrate comprises a HSV comprising a hue of from about 0 to about 80, a saturation of from about 0 to about 25%, and/or a value of from about 0% to about 50%.

10. The DMF cartridge of claim 1, wherein the colored coverlay substrate comprises multiple colors on a surface of the colored coverlay substrate, and wherein each of the multiple colors are selected from white, gray, silver, light yellow, and black.

11. The DMF cartridge of claim 10, wherein the multiple colors are sectioned on the surface of the colored coverlay substrate according to the assay being performed on the DMF cartridge.

12. The DMF cartridge of claim 10, wherein the multiple colors are sectioned on the surface of the colored coverlay substrate to correspond to functions performed on the top plate and bottom plate.

13. The DMF cartridge of claim 12, wherein each of the functions performed on the top plate and the bottom plate correspond to a color of the multiple colors.

14. The DMF cartridge of claim 10, wherein the surface of the colored coverlay substrate comprises one or more regions.

15. The DMF cartridge of claim 14, wherein the one or more regions each comprise one or more of the multiple colors.

16. The DMF cartridge of claim 1, wherein the colored coverlay substrate comprises a multiple color coverlay substrate, and wherein the multiple colored coverlay substrate comprises a white section and a black section.

17. The DMF cartridge of claim 1, wherein the droplet operation gap between the top plate and the bottom plate is filled with a filler fluid.

18. The DMF cartridge of claim 17, wherein the filler fluid is a low-viscosity oil or a halogenated oil.

19. A method for analyzing an analyte of interest using an optical detection operation, the method comprising:
(a) providing a digital microfluidic (DMF) cartridge, where the DMF cartridge comprises:
(i) a top plate, wherein the top plate comprises a top plate substrate;
(ii) a bottom plate, wherein the bottom plate comprises a colored coverlay substrate having a plurality of electrodes operable to perform droplet operations; and
(iii) a droplet operation gap between the top plate and the bottom plate;
(b) providing a sample droplet to be analyzed for the presence of the analyte of interest;
(c) performing droplet operations on the droplet sample, wherein the performing comprises moving the droplet sample to a sensor well in the DMF cartridge;
(d) performing an optical detection operation by illuminating the droplet sample in the sensor well using an illumination source;
(e) detecting light emitting from the droplet sample using an optical measurement device; and
(f) using a processor, detecting the presence of an analyte and/or determining the concentration of the analyte by analyzing the detected light.

20.-37. (canceled)

38. A method for detecting an analyte using colorimetric-based optical detection operations, the method comprising:
(a) providing a digital microfluidic (DMF) cartridge, where the DMF cartridge comprises:
(i) a top plate, wherein the top plate comprises a top plate substrate;
(ii) a bottom plate, wherein the bottom plate comprises a colored coverlay substrate having a plurality of electrodes operable to perform droplet operations, wherein the color of the colored coverlay substrate is white, light gray, silver or light yellow; and
(iii) a droplet operation gap between the top plate and the bottom plate;
(b) providing a sample droplet to be analyzed for the presence of an analyte of interest;
(c) performing droplet operations on the sample droplet to move the sample droplet to a sensor well in the DMF cartridge;
(d) performing a colorimetric-based optical detection operation by illuminating the sample droplet using a colorimetric-based illumination source;
(e) detecting light emitted from the sample droplet at one or more color specific wavelengths using an optical measurement device; and
(f) using a processor, detecting the presence of an analyte and/or determining the concentration of the analyte by analyzing the detected light.

39.-58. (canceled)
